(19) United States
(12) Patent Application Publication
(10) Pub. No.: US 2021/0268039 A1
(43) Pub. Date: Sep. 2, 2021

(54) USE OF LEUCONOSTOC MESENTEROIDES SUBSP. MESENTEROIDES SD23 FOR MATERNAL FETAL METABOLIC PROGRAMMING

(71) Applicant: INSTITUTO NACIONAL DE CIENCIAS MÉDICAY Y NUTRICIÓN SALVADOR ZUBIRÁN, Ciudad de México (MX)

(72) Inventors: Diana Catalina CASTRO RODRÍGUEZ, Ciudad de México (MX); Elena ZAMBRANO GONZALEZ, Ciudad de México (MX)

(21) Appl. No.: 17/258,195

(22) PCT Filed: Jul. 11, 2019
(86) PCT No.: PCT/IB2019/055932

§ 371 Date: Jan. 6, 2021

(30) Foreign Application Priority Data
Jul. 13, 2018 (MX) MX/A/2018/008663

(51) Int. Cl. A61K 35/744; A61P 3/04; A61P 15/08

(52) U.S. Cl. CPC A61K 35/744; A61P 15/08; A61P 3/04

(57) ABSTRACT

Implemented new use of probiotics, especially of the Leuconostoc mesenteroides subsp. mesenteroides SD23 strain to be consumed during pregnancy and lactation of obese mother and to partially or totally prevent metabolic alterations caused by a high fat diet both in the mother and the offspring.

### FIELD OF THE INVENTION

This invention is related to the field of compositions with nutraceutical or therapeutic properties, specifically to the compositions to be used in the treatment or prevention of obesity and overweight in the offspring of obese mothers, where the composition administered during pregnancy comprises a strain of Leuconostoc mesenteroides subsp. mesenteroides SD23 as active ingredient.

### BACKGROUND OF THE INVENTION

Obesity is a public health problem in Mexico affecting different groups of people in a large or lower extent. Although the main reason causing this disease is evidently an unbalance between intake and the energy output, there is current evidence indicating that a combination of diet, environment and genetic factors are also a reason for this type of pathology [1-3]. Such studies state that the composition of the microbiota of every individual is a determining factor favoring the obesity or a poor phenotype.

Women of reproductive age and school age children are found in the sectors of the population suffering obesity. Different researches about child obesity indicate that this disease may also be the result, not only from the sedentariness, life style and food conditions, but also from the nutritional and metabolic conditions of the mother. It has been noted that the maternal obesity predisposes the growing fetus and neonate to the development of metabolic diseases from childhood that continue in the adult life. Characteristics of the metabolic syndrome such as insulin resistance, hyperglycemia, increase of the inflammatory process, increase of the oxidative stress and change in the intestinal microbiota [3-5] are found in the adverse effects caused by maternal obesity to the progeny. Based on the evidence of the effects of the mother on the child, the hypothesis “developmental origins of health and disease”, DOHaD, has arisen that proposes that the fetal and neonatal physiology and metabolism may be altered by changes during a critical time window of the development, namely, during gestation and lactation. These alterations produce a physiological response in the fetus associated with the development of diseases in the adult [6, 7]. The fetus and neonate metabolically scheduled present permanent modifications on the structure and physiology of organs and on the expression of genes involved in their own metabolism [8]. Therefore, the phenotype of the adult is the total of the genetic factors and of the influence of the fetal and postnatal environment. It has been reported that the development of obesity before and during pregnancy is a factor responsible for the adverse effects of the programming of the development of the offspring, such as predisposition to diabetes, increase of abdominal adipose tissue, obesity and cardiovascular diseases [9-12].

As above-mentioned, microbiota is a factor implied in the obesity and in the associated diseases, due to its influence on the metabolic and immunological functions of the host. Newborns are rapidly colonized by different bacteria during their first days of life, to which the initiation of the defense system is attributed, thus guaranteeing good physical and immunological development [13]. This invasion of the digestive tract of the neonate occurs through the placenta, the amniotic liquid, birth condition and lactation that determine a healthy or altered microbiota from which different diseases are derived. Several studies have reported that the maternal immune cells and the bacteria of the intestinal tract of the mother cross the placenta and modulate the immune responses on the fetus [8-14]. It has also been demonstrated that microbiota of the mammary gland is unique and beneficial bacteria are found that access to the gland through an internal route and that are transferred to the neonate once the lactation begins, thus producing protection factors for its future life [8].

If it is true that pregnancy is a vulnerability period for predisposition to diseases in the postnatal life, it is also true that it is a window of opportunities to implement interventions aimed to improve health of pregnant woman and therefore, her descendants. Several studies in animal models evidence that the nutritional or exercise intervention on the pregnant obese mother totally or partially prevents the offspring from the adverse effects of programming [7, 15, 16].

Another type of therapeutic intervention explored during the recent years is the use of probiotics as supplements of nutritional diets. Studies have mainly focused on the classes of Lactobacillus and Bifidobacterium, such effects are summarized in Table 1.

Evidence of the use of probiotics for the treatment of different pathologies has evidenced that the effects depend on the bacterial strain and on the characteristics of the host such as the age and nutritional condition [17].

If it is true there are not many works reporting the use of isolated probiotics in the treatment and/or prevention of complications during pregnancy, lactation and health of offspring of mothers with metabolic alterations, as with the other therapeutic uses of probiotics, it is impossible to predict that a previously disclosed specific effect of a strain will be the same in other strain of the same kind but without known effects, for the treatment of the same disease.

In other words, an expert-in-the-art of probiotics is aware there are specific effects of the different bacterial strains inclusively in one same kind; based on the aforementioned, it is important that the studies are performed at bacterial strain level and never to transfer the obtained effects from strain to strain in the same kind [18].

In addition to the aforementioned complications, many of the controlled interventions in adults are not viable during pregnancy and this adds a higher level of uncertainty to a strain in mothers and in their descendants.

Evidence related to the maternal probiotic interventions summarized in Table 1 shows that there is no evidence in the state-of-the-art as of now of the use of the bacterial kind Leuconostoc in the pregnant mother or in her offspring.

As a matter of fact, there are few patent documents evidencing specific effects of Leuconostoc mesenteroides on adult individuals, since the Leuconostoc mesenteroides is usually administered as a mixture along with other strains and inclusively bacterial kinds as in US20140079676 (Omstead, 2014); or a mixture to produce a probiotic composition which inoculant includes the Leuconostoc mesenteroides among other bacterial kinds as in US20080064657 (Day et al, 2008) and WO2017146213 (Kimura, 2017); or the use of the Leuconostoc mesenteroides is inclusively mentioned only as part of the inocolum to ferment food as the kimichi KR20120021349 (Sang Kyu, 2012) where such ferment is the direct responsible of the therapeutic effects disclosed in such document.

TABLE 1
Beneficial effects of disclosed probiotic
strains during pregnancy and lactation.

| Experi- | | | |
|---|---|---|---|
| mental | | | |
| Model | Strain | Health Benefits | Ref. |
| Human | Lactobacillus rhamnosus | Supplements with | 19 |
| | GG (ATCC53103)/ | these probiotic bacteria | |
| | Bifidobacterium lactis | at the beginning of | |
| | | pregnancy are effective | |
| | | to control weight and | |
| | | body composition of | |
| | | the mother during and | |
| | | after pregnancy. | |
| Human | VSL#3 (Streptococcus | In women with | 20 |
| | thermophilus, | gestational diabetes | |
| | Bifidobacterium breve, | mellitus, intervention of | |
| | Bifidobacterium lungum, | mixture VSL#3 | |
| | Bifidobacterium infantis, | regulates some | |
| | Lactobacillus acidophilus, | inflammatory markers | |
| | Lactobacillus plantarum, | and it had benefits on | |
| | Lactobacillus paracasei/ | the glycemic control in | |
| | Lactobacillus delbrueckiil | pregnant women. | |
| | subsp. Bulgaricus) | | |
| Human | Lactobacillus rhamnosus | Reduction of | 21 |
| | HN001 | gestational diabetes | |
| | | mellitus prevalence, | |
| | | especially in older | |
| | | women. | |
| Human | Lactobacillus acidophilus/ | Probiotic intervention | 22 |
| | L. casei/ | in pregnant women | |
| | Bifidobacterium bifidum | with gestational | |
| | | diabetes mellitus had | |
| | | beneficial effects on | |
| | | the glycemic control, | |
| | | triglycerides and VLDL | |
| | | cholesterol | |
| | | concentrations. | |
| Human | Lactobacillus acidophilus/ | Consumption of | 23 |
| | Bifidobacterium lactis/ | probiotics is associated | |
| | Lactobacillus rhamnosus | with a lower risk of | |
| | GG | pre-eclamsia in first- | |
| | | time mothers. | |
| Human | Lactobacillus rhamnosus | Administration of | 24 |
| | LPR/Bifidobacterium | probiotics to the | |
| | longum BL999/L paracasei | pregnant mother and | |
| | ST11 | during lactation period | |
| | | is safe and effective to | |
| | | reduce the eczema risk | |
| | | on babies. | |
| Human | Lactobacillus rhamnosus | Intervention with | 25 |
| | GG and Bifidobacterium | probiotics during | |
| | lactis Bb12 | pregnancy decreased | |
| | | the glucose | |
| | | concentration in the | |
| | | blood of the diabetic | |
| | | mothers. | |
| Mouse | L. paracasei NCC 2461 | Maternal probiotic | 26 |
| | | intervention prevented | |
| | | the development of | |
| | | inflammation of the | |
| | | respiratory airways in | |
| | | the offspring. | |

As per the above-described, consumption of probiotics plays a significant role on maintenance of the intestinal ecosystem and on the stimulation of the immune system, improving or preventing certain pathologies, inclusively pathologies related to pregnancy and lactation and their effects on the offspring. That is why, this invention proposes the novel use of a probiotic strain of Leuconostoc mesenteroides to be consumed during pregnancy and lactation in the obese mother and to partially or totally prevent metabolic alterations caused by a high fat diet both on the mother and on the offspring.

### BRIEF SUMMARY OF THE INVENTION

Purpose of this invention is to implement a new use of the probiotics, especially of the Leuconostoc mesenteroides subsp. mesenteroides SD23 strain to be consumed during pregnancy and lactation of the obese mother and to partially or totally prevent metabolic alterations caused by a high fat diet both on the mother and the offspring.

It is shown in an embodiment of the invention that the administration of the Leuconostoc mesenteroides subsp. mesenteroides SD23 improves the fertility rate of female rats fed with a high fat diet.

In another embodiment of the invention, administration of the Leuconostoc mesenteroides subsp. mesenteroides SD23 decreased the serum concentration of glucose, cholesterol and triglycerides of female rats that consumed a high fat diet during the pregnancy and lactation periods.

Administration of the Leuconostoc mesenteroides subsp. mesenteroides SD23 strain is another characteristic of the invention to reduce length of the small intestine villi that are altered by the high fat diet.

It is shown in an embodiment of the invention that the maternal intervention with the Leuconostoc mesenteroides subsp. mesenteroides SD23 strain prevents accumulation of fat in the offspring of the obese mothers.

A last embodiment shows that the maternal intervention with Leuconostoc mesenteroides subsp. mesenteroides SD23 strain reduces the triglyceride levels on the male offspring of obese mothers.

### BRIEF DESCRIPTION OF FIGURES

FIG. 1. Effect of probiotic consumption on the fertility rate.

FIG. 2. Weight before pregnancy and maternal weight during pregnancy and lactation in presence and absence of probiotic.

FIG. 3. Effect of probiotic consumption on the biochemical parameters of mothers with 21 days of lactation.

FIG. 4. Effect of probiotic consumption on the small intestine histology of mothers with 21 days of lactation.

FIG. 5. Effect of probiotic consumption on the weight of the offspring during puberty (36 days postnatal).

FIG. 6. Effect of probiotic consumption on the biochemical parameters of offspring during puberty (36 days postnatal).

FIG. 7. Effect of probiotic consumption on the body composition of the offspring during young adult age (110 days postnatal).

FIG. 8. Effect of probiotic consumption on the biochemical parameters of offspring during young adult age (110 days postnatal).

FIG. 9. Effect of probiotic consumption on the body composition by magnetic resonance on the offspring at the mature adult age (350 days postnatal).

### DETAILED DESCRIPTION OF THE INVENTION

SD23 strain corresponding to the genus and species Leuconostoc mesenteroides presents probiotic properties comparably more favorable than other strains of the same species Leuconostoc and other genus of Lactobacillus and Bifidobacterium strains, bacteria commercially considered as probiotic. This invention addresses a way of favorably intervening on the metabolic disorders caused by the maternal obesity to the offspring throughout their lives, this study has not been described by any strain of the Leuconostoc genus disclosed as of now. The most interesting fact is that any of the disclosed strains of this genus or species has shown to be useful for the simultaneous and effective treatment of the metabolic disorders associated to the maternal obesity and its descendants. Therefore, this invention provides a new strain of the Leuconostoc mesenteroides species with high value for the treatment of maternal obesity.

The reason why the Leuconostoc mesenteroides subsp. mesenteroides SD23 strain is of great interest to reach a solution of the metabolic disorders caused by obesity is described here in below.

1. The administration of the Leuconostoc mesenteroides subsp. mesenteroides SD23 strain improves the fertility rate of female rats fed with a high fat diet. This finding is of great importance, since some of the problems an obese mother in reproduction age faces is the low fertility rate due to the disorders presented by the high fat diets.

2. Administration of the Leuconostoc mesenteroides subsp. mesenteroides SD23 strain decreases the serum concentrations of glucose, cholesterol and triglycerides in female rats that consumed a high fat diet during the pregnancy and lactation periods.

3. Leuconostoc mesenteroides subsp. mesenteroides SD23 strain reduces the length of small intestine villi that are altered by a high fat diet, thus indicating that it may reduce the fat absorption of the diet and it may be a mechanism by means of which this strain has a positive effect on the decrease of concentrations of cholesterol and triglycerides. Another benefit of this strain at intestine level is to prevent hyperplasia of the goblet cells affected by obesity.

4. Maternal intervention with the Leuconostoc mesenteroides subsp. mesenteroides SD23 strain prevents the offspring of obese mothers during puberty age (36 days postnatal), young adult ages (110 days postnatal) and mature adult (350 days postnatal) both in males and females from accumulation of fat and a decrease of weight, total fat and adiposity rate is noted. Reduction of total fat is clearly noted on the decrease of gonadal fat and this allows inferring it will help on the reproduction system of males.

5. Maternal intervention with the Leuconostoc mesenteroides subsp. mesenteroides SD23 strain reduces the triglycerides concentration on the male offspring of obese mothers during young adult age.

It is important to highlight that the intervention with the Leuconostoc mesenteroides subsp. mesenteroides SD23 strain does not produce any alteration on the health control of rat mothers during the pregnancy and lactation periods, namely, no adverse effects are observed such as abdominal inflammation or diarrhea; hence, administration of the Leuconostoc mesenteroides subsp. mesenteroides SD23 strain is not dangerous even when the administration is during high risk periods for the development of the descendants, pregnancy and lactation.

### Characteristics of the Microorganism

Molecular sequence 16S rRNA is stored in the GenBank database with the access number KR476473.
Scientific classification of the SD23 strain of this invention is: Kingdom: Bacterial Phylum: Firmicutes/Order: Lactobacillales/Family: Leuconostocaceae/Category: Leuconostoc/Species: mesenteroides/Subspecies: mesenteroides.
Substrates that bacteria SD23 oxides or ferments are: L-Arabinose; D-Ribose; D-Xilose; D-Galactose; D-Glucose; D-Fructose; D-Manose; D-Manitol; Methyl-αD-Glucopiranose; N-Acethylglucosamine; Amigdaline; Arbutine; Ferric citrate esculin; Salicine; D-Celobiose; D-Maltose; D-Lactose (bovine origin); D-Melibiose; D-Sucrose; D-trehalose; D-Rafinose; Gentiobiose; D-Turanose; potassimum 2-Ketogluconate.
Strain grows in a temperature interval from 28-37° C. with an optimum 30° C.
Strain grows in a pH interval from 5-7, with an optimum pH of 6.5.
In addition, strain is viable at gastrointestinal conditions (acid pH and high concentration of bile). Its viability at stomach conditions (pepsin 3 g/l at pH 2 and during 2 h) is 46.51% and at intestinal conditions (bile salts 0.5%, pH 6.5 and pancreatin 1.9 g/l) is 89.36%. It is also resistant to the conditions of preservation processes (freezing, lyophilization).
Strain is isolated from a native source of Mexico, the “aguamiel” of Agave salmiana.

The following definitions are provided herein for ready reference:

“Probiotic” means the live microorganism more specifically probiotic bacteria in specific amounts scientifically studied in connection with beneficial effects on human health when they are consumed; even more specifically such beneficial effects are due to an improvement in the balance of the intestinal microbiota.

“Prebiotics” means non-digestible polysaccharides specifically promoting the selective stimulation of growth and/or activity or activities of one or more microbial species and in a limited way of the intestinal microbiota that grants a benefit to the host. Therefore, the probiotics are referred to any non-viable food component specifically fermented in the colon by native bacteria that are believed of a positive value, for example, Bifidobacterium, Lactobacillus, etc.

“Symbiotic” means the combined administration of a probiotic strain with one or several prebiotic compounds that may improve the in vivo probiotic growth resulting into a higher benefit to the health and it is denominated symbiotic.

“Intestinal microbiota” means the group of microorganism (bacteria, archaea, yeast, unicellular eukaryotic cells and helminth and virus) found throughout the gastrointestinal system of a host.

“BMI” or “Body Mass Index” means, a weight measure of a person at scale with the height, specifically the body weight of an individual divided into the square of his/her height (medium weight in kilograms, height in meters). The formula universally used in medicine produces a measurement unit of kg/m². According to the US Department of Health & Human Services, a BMI under 18.5 indicates thinness, 18.5-24.9 normal weight, 25-29.9 overweight and a BMI of 30 or above indicates obesity.

“Overweight” means a 25-29.9 BMI.

“Obesity” means a 30 or above BMI.

“Weight gain” means the increase of weight resulting from an excessive diet intake comprising an excessive fat diet intake especially unsaturated fat and, as option, an excessive diet intake of simple carbohydrates. For a given experimentation subject, an excessive diet intake, especially of fat and as option of simple carbohydrates, above the necessary amount to satisfy the physiological needs and keep the energy balance of the experimentation subject, the effect of a treatment over the reduction or prevention of body weight gain and metabolic parameters related to obesity, described in detail in the scientific literature, induced by a diet in an experimentation subject may be evaluated by means of comparison of the body weight gain observed in an experimentation subject receiving the treatment versus those parameters observed in the same experimentation subject to whom the treatment is not provided and that receives the same diet and that has the same level of physical activity.

“Reduce the weight gain” means limiting, reducing or decreasing in a subject, more specifically in an experimentation subject, the improvement of the body weight induced by a treatment versus the improvement of the body weight induced by the diet in the experimentation subject but that he/she/it did not consume due the treatment.

“Treatment” for obesity or weight gain or “treating” obesity or weight gain means in this invention, the purpose of decreasing or keeping the body weight of a subject.

“Prevention” means administration of the composition of this invention against obesity to reduce or keep the body weight of a subject in risk of obesity. In an embodiment, prevention involves keeping the body weight of the subject immediately before beginning of administration of the composition against obesity.

“Food supplement” means a product manufactured as from compounds usually used in foods but in the form of tablets, powders, capsules, potions or in any other form that is not generally associated to food and that has beneficial effects for the health of the person.

“Functional food” means food also presenting beneficial effects for the health of a person. More specifically, functional food has a beneficial physiological effect over a chronic disease.
“Nutraceutical” means pharmaceutical grade standardized nutrient, more specifically they are products derived from food sources which purpose is providing extra healthy benefits to the nutritional values found in food. Such nutraceutical products, depending on the jurisdiction may have properties for prevention of chronic diseases or health improvements.

Preferred forms but not limiting forms are described here in below:

In one embodiment, the composition of this invention may be comprised only of the Leuconostoc mesenteroides subsp. mesenteroides SD23 strain or it may be a food composition or pharmaceutical composition containing said strain and other optional components. There are no specific limitations in these optional components provided they are pharmaceutical or food grade and that they comply with the sanitary rules and regulations provided by the corresponding authorities.

In one embodiment of the composition of the invention, the strain of this invention may be used in entire bacteria form, more specifically in the form of a bacterial lysate, preferably the bacterial strain may be in the form of live cells.

When the Leuconostoc mesenteroides subsp. mesenteroides SD23 strain is alone or in composition with prebiotics or other bacterial species strains, it is in the form of live bacteria, the composition may have from 1×10⁵ to 1×10¹³ colony forming units (CFU), preferably a minimum of 1×10⁶ CFU, more preferably minimum, 1×10⁷ CFU; even more preferably minimum, 1×10³ CFU; and mostly preferred minimum 1×10¹⁰ CFU per dried weight gram of the composition. In the case of the liquid composition, this corresponds, in general from 1×10⁴ to 1×10¹² CFU/ml, preferably minimum 1×10⁵ CFU/ml, more preferably minimum from 1×10⁶ CFU/ml, even more preferably minimum from 1×10⁷ CFU/ml and mostly preferred minimum of 1×10¹⁰ CFU/ml. Such above-mentioned amounts may be administered in a daily intake or in more than once, during consumption of a high fat diet.

Determination of an applicable effective amount in humans could be inferred by an expert-in-the-art, especially in view of the detailed disclosure stated in this description.

Examples of the form of dosage of the Leuconostoc mesenteroides subsp. mesenteroides SD23 strain of the composition of this invention, in the case of oral administration include solid formulations such as powder, granules, tablets and capsules and liquid formulations as syrups, suspensions and emulsions without excluding semi-solid forms or powders. In addition, in the case of parenteral administration, examples of dosage include suppositories, ointments, sprays or nebulizers.

In an embodiment of this invention, it may be administered either orally, by colposcopy, by rectum by means of a suppository. Oral administration of the composition is preferred which is comprised of the bacterial strain according to this invention, more preferably in the form of capsules, tablets, powders, granules or solutions or oral suspensions.

Production of these formulations may be made using conventional methods, according to the dosage form.

During the production of these formulations, they may be comprised only of the active ingredient or it may also include a suitable pharmaceutical carrier.

In those cases where a pharmaceutical carrier is added, there are no specific limitations in the amount of mixture of the active ingredient, namely, the Leuconostoc mesenteroides subsp. mesenteroides SD23 strain of this invention, where such amount may be suitably determined according to the dosage form, such a powder, tablet or capsule as long as the bacteria content is in the interval from 1×10⁶ to 1×10¹² CFU/g, mostly preferably a minimum of 1×10¹⁰ CFU/ml.

Any conventionally acceptable organic or inorganic carrier may be the pharmaceutical carrier, depending on the dosage form. In the case of solid formulations, examples of carrier include excipients, binders, disintegrants, lubricants, stabilizers and correction agents and the like.

Composition of this invention comprises food products, food supplements, nutraceutical products, nutritional supplements, medical food or functional food.

Food or beverage of this invention against obesity may be produced by using the Leuconostoc mesenteroides subsp. mesenteroides SD23 strain against obesity and other acceptable components to include them as raw materials in the food or beverages or they may be produced upon adding the Leuconostoc mesenteroides subsp. mesenteroides SD23 strain of this invention against obesity to existing food or beverages containing other components.

Composition of the invention may be a dairy product, preferably, a fermented dairy product, the fermented product may be present in the form of a liquid or present in the form of dried powder obtained by drying of the fermented liquid, more specifically, it may be in the form of dairy products including fermented milk and/or curd wheycurdied, shaked, drinkable such as cheese or yoghurt.

Fermented product may also be vegetable, such as soya, tubers, cereal or fruits.

Composition may be presented in the form of a product derived from milk, especially, fermented milk comprising at least the Leuconostoc mesenteroides subsp. mesenteroides SD23 strain as combined option, such as it was previously mentioned, with other bacteria of the Lactobacillus genus, for example, in the form of fermented food.

There are no specific limitations in the other components included in the food or beverage, as long as the components have been tested for addition in food and beverages according to the regulations of food such as the Food Sanitation Act or the Federal Commission for Protection against Sanitary Risks (COFEPRIS for its initials in Spanish) or the regulation corresponding to the territory in question and they do not alter the effect on the decrease of weight gain in subjects with a high fat diet of such composition.

There are no specific limitations in the form of food or beverage that includes any form of edible composition of Leuconostoc mesenteroides subsp. mesenteroides SD23 strain and an acceptable carrier for inclusion in food and beverages. Specific examples include all forms of food or beverages including solid food such as bread, chewing gum, cookies, chocolate, candies and cereals, jelly-based food, cream-based and gel-based such as jelly, ice cream, yoghurt and jellow and beverages such as juices, coffee and cocoa. In addition, flavoring and food additives and the like may be added.

Food or beverage against obesity containing the Leuconostoc mesenteroides subsp. mesenteroides SD23 strain according to the dosage of this invention, is preferably commercialized as food or beverage indicating its use for applications against obesity, it is worth highlighting that the expression used for the above-described type of indications is not limited to the phrase “against obesity” and it is not necessary to say it, any other expressions indicating an effect against obesity are also included in the scope of this invention. In addition, the food or beverage may also be commercialized in a similar way as food or beverage indicating its use to prevent or ameliorate lifestyle disease for which obesity is a known risk cause or factor, such as hyperlipidemia, hypertension and diabetes.

This invention may be more clearly understood as from the following additional description with examples that enable to better understand but not limited to any other embodiment of the invention.

### Methodology

Maternal obesity model: An obesity condition was induced (MO) in Wistar line recently weaned female rats (F0), with an obesogenic diet that contains 23.5% protein, 20.0% lard, 5.0% corn oil, 20.2% polysaccharides, 20.2% simple sugar, 5.0% fiber, 5.0% mixture of minerals and 1.0% mixture of vitamins (w/w), energetic value is 4.9 kcal/g; that was provided as from wean during growth and until mating (120 days), pregnancy and lactation. The control group (C) was fed during the same period with a commercial diet for rodents (Zeigler Rodent RQ22-5) containing 22.05% protein, 5.0% vegetable oil, 31.0% polysaccharides, 31.0% simple sugar, 4.0% fiber, 6.0% minerals and 1.0% vitamins (w/w), energetic value is 4.0 kcal/g.
Maternal interventions: Half of MO females were intervened as from 90 days (namely, a month before pregnancy) until mating and during all pregnancy and lactation with the probiotic bacteria Leuconostoc mesenteroides subsp. mesenteroides SD23. Groups were identified as CP and MOP. Control group (C) and obese group (MO) continued with the commercial diet and the high fat diet, respectively throughout the study.

Groups and intervention of the probiotic strain are shown in detail here in below:
Control group (C): females were fed with the commercial diet for rodent and a daily volume of 0.1 mL of milk at 10% was administered.
Probiotic control group (CP): females were fed with the commercial diet for rodent and a daily volume of 0.1 mL at a 1×10¹⁰ CFU/mL concentration of L. mesenteroides SD23 was administered.
Obese group (MO): females were fed with a high fat diet and a daily volume of 0.1 mL of mil at 10% was administered.
Probiotic obese group (MOP): females were fed with a high fat diet and a daily volume of 0.1 mL at a 1×10¹⁰ CFU/mL concentration of L. mesenteroides SD23 was administered.
Mating of females (F0): Females were mated with non-experimental males at 120 days of age. Rats had natural birth.
Obtaining of biological samples: sternum, pancreatic, retroperitoneal, mesenteric and gonadal fat of the mothers (21 days lactation) and from the offspring at 36 and 110 postnatal days was collected and individually weighed to calculate the adiposity index (total adipose tissue (g)/body weight (g)). Length of intestinal villi in the mothers was analyzed as effect from the high fat diet and from intervention with the probiotic.
Analysis of biochemical parameters: Serum concentrations of glucose, triglycerides and cholesterol were analyzed by means of an enzymatic method using the automatic Synchron X analyzer.
Statistical analysis: All data was reported as the ±EE average. Results were analyzed by one-way ANOVA with the Tukey test, p<0.05.

Results

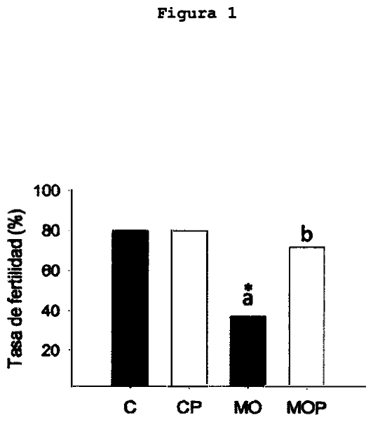

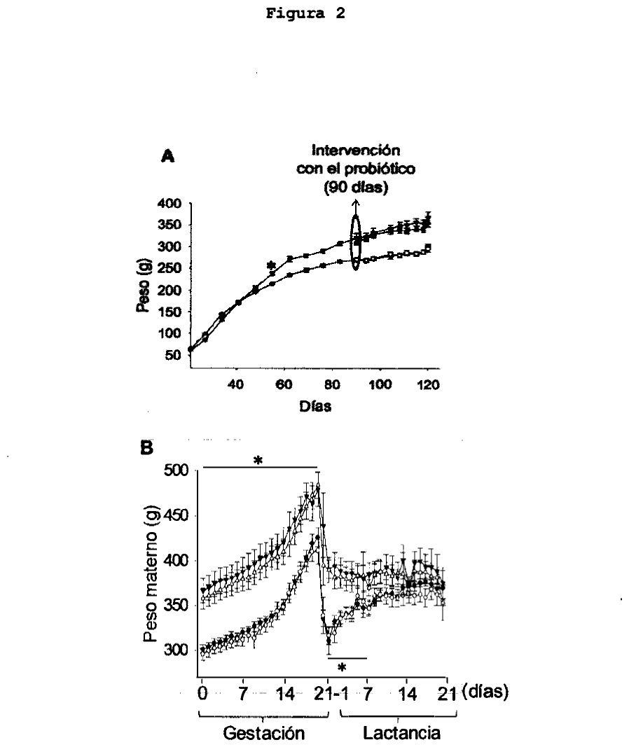

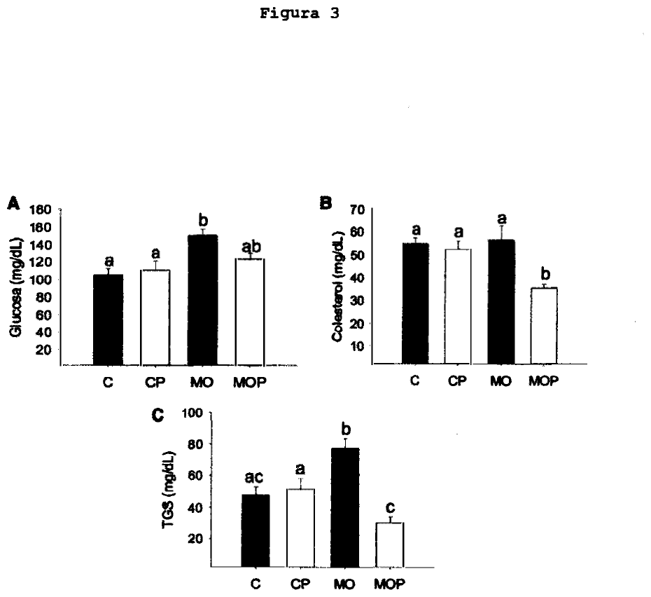

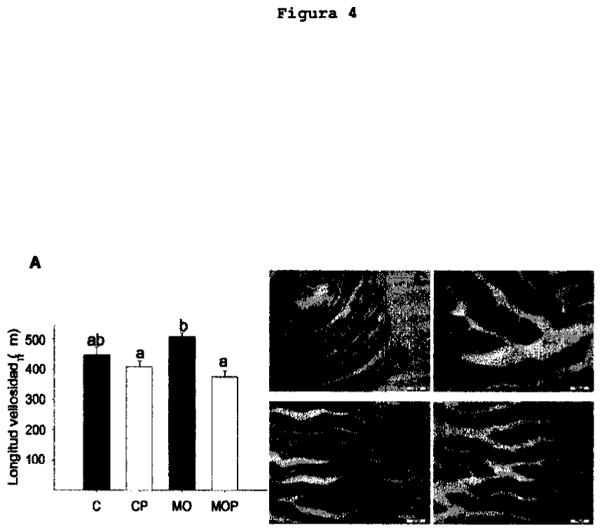

Prior, Pregnancy and Lactation:
Obesity decreased the fertility rate in the MO group but intervention with the probiotic in the MOP increased to 35% the fertility rate in comparison with MO. Fertility percentage in C and CP mothers was 80% (FIG. 1). Weights of rats used for the maternal obesity model at 21 days from beginning the experiment were similar in group C and MO but it was noted on day 55, 34 days after the beginning of the high fat diet that the weight is significantly different between C and MO. Intervention with the probiotic started at 90 days (FIG. 2A). This intervention continued during pregnancy and lactation (FIG. 2B). Maternal weight was not affected by the intervention with the probiotic and during all stages the control groups (C and CP) presented less weight in comparison with the groups fed with high fat diet (MO and MOP). By the end of pregnancy and as from the seventh day of lactation, weights of four groups did not present significant differences.
Maternal biochemical parameters at the end of lactation: Glucose, cholesterol and TGS concentrations of the four experimental groups (C, CP, MO and MOP) were observed in FIG. 3. MO presented the highest values of glucose and TGS in serum in comparison with C and MOP, problems of a high fast diet were observed. While the administration of Leuconostoc mesenteroides subsp. Mesenteroides SD23 prevents increase of glucose and TGS concentrations in the MOP. Concentrations of cholesterol were similar in C, CP and MO; in MOP were under compared to control.
Maternal analysis in the small intestine at the end of lactation: Histological analysis of the intestine permitted to observe the effect of the administration of Leuconostoc mesenteroides subsp. mesenteroides SD23 strain on the maternal obesity. Length of intestine villi may be noted in FIG. 4, a reduction of villi length in CP and MOP may be observed. Feeding with a high fat diet alters the intestinal morphology; resulting an increase of intestinal villi length and the expression and release of intestinal peptides controlling the intake of food (Covasa 2010; Mao et al., 2013). On the other hand, alterations in the intestine may be the result from increase in the absorption of fat. High fat feeding may also cause hyperplasia in the globet cells present in the intestinal wall (Covasa 2010). Main function of intestinal globet cells is the formation of mucin useful as defense mechanism of host (Kim and HO, 2010). Mucin synthesis in the intestinal globet cells occurs as response to a series of stimulations, hormonal influence, inflammatory factors, bacterial toxins and chemical factors (Specian et al., 1991). Hyperplasia of globet cells is accompanied with defective mucin synthesis and this enables that the pathogens may invade and attach to the intestinal surface. Therefore, this invention shows that the administration of the Leuconostoc mesenteroides subsp. mesenteroides SD23 strain has an effective effect on decreasing fat absorption at an intestinal level.

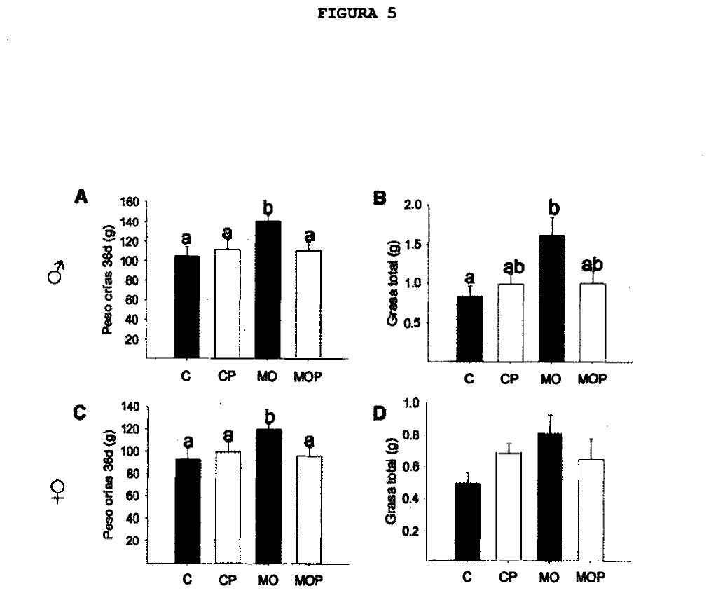

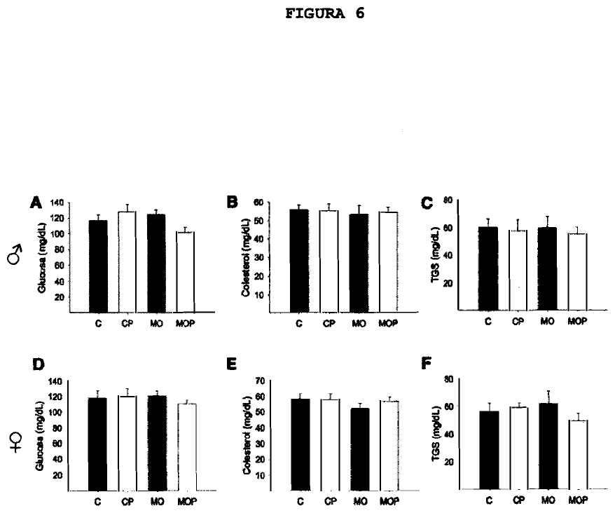

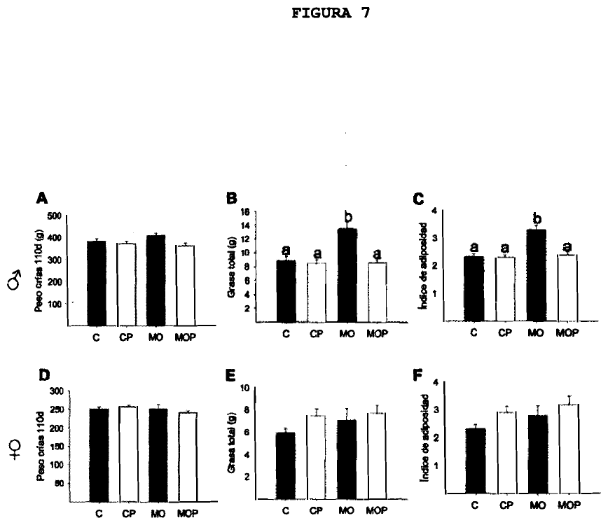

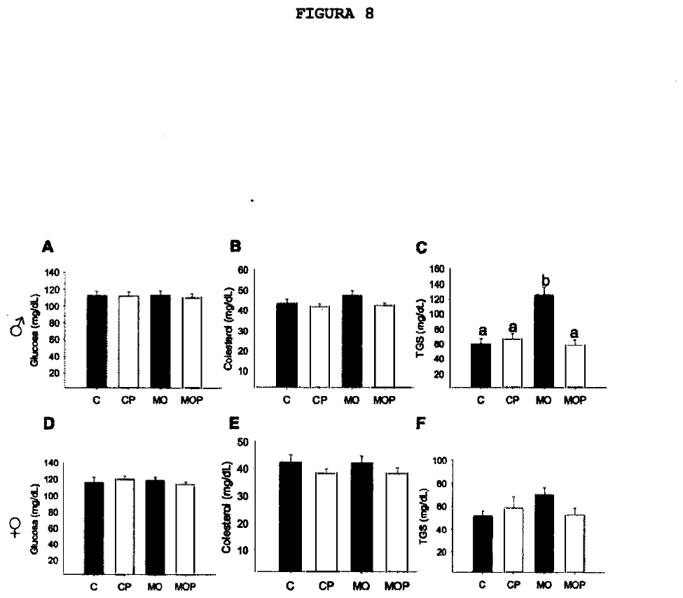

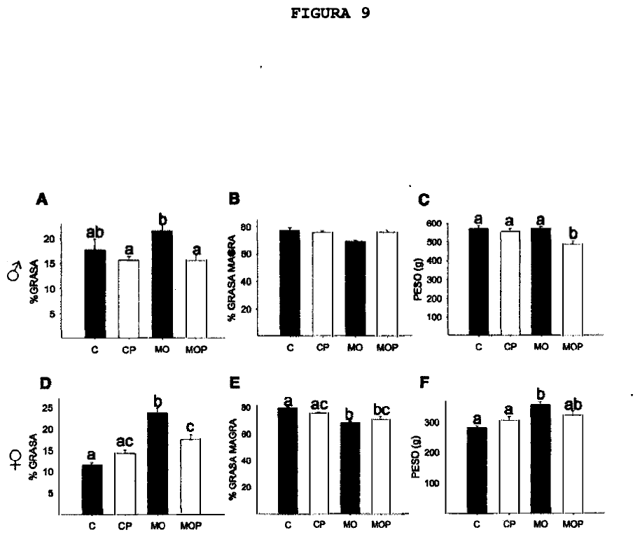

Results in Offspring of 36 and 110 Days
Maternal obesity programs the offspring to develop obesity during its postnatal life, this is observed in FIG. 5, where it is observed that the offspring in puberty (36 days postnatal), descendants of obese mothers (MO) presented higher body weight in comparison with the offspring of mothers intervened with the probiotic (MOP). It may also be noted that the total fat in the females does not present a significant difference among the four experimental groups. Regarding the biochemical parameters, the offspring is not present at 36 days significant differences among the four experimental groups (FIG. 6).
   The type of programming to which the offspring is exposed during the pregnancy and lactation period affects its development at young age (110 days postnatal), as it may be noted in FIG. 7; despite the offspring presents the same body weight, its composition in total fat shows once more that the offspring from MO presented at 110 days more fat and adiposity index in comparison with C, CP and MOP. Higher effect on male offspring is noted. Glucose, cholesterol and TGS concentrations may be noted in FIG. 8, female offspring did not present significant differences at 110 days among the four experimental groups, in turn, TGS concentrations increased in the male offspring from obese mothers (MO) but the maternal intervention with the probiotic decreases the TGS concentrations in its offspring (MOP). Type of diet and intervention to which the mother was subject during the pregnancy and lactation periods program its offspring to develop obesity or metabolic problems during its postnatal life. It is noted in FIG. 9 that the offspring at a mature adult age (350 days postnatal) continue presenting effects of the diet or maternal intervention. Offspring from obese mothers (MO) has greater percentage of fat in comparison with the offspring of mothers intervened with the probiotic (MOP). Results obtained by the maternal intervention with the probiotic Leuconostoc mesenteroides subsp. mesenteroides SD23 have beneficial effects both on the maternal metabolism as on its offspring at different postnatal ages. This intervention is an opportunity to prevent or improve the problems caused by a negative programming due to the type of diet that to which the mother has been exposed during pregnancy and lactation.

### REFERENCES

1. Schnitzler M., et al., (2012). A role for microchimerism in obesity and evolution. Med. Hypotheses. 78 (4): 528-532.
2. Angelaski E., et al., (2012. The relationship between gut microbiota and weight gain in human. Future Microbiol. 7 (1): 91-99.
3. Zambrano, E., et al., (2016). Maternal obesity: lifelong metabolic outcomes for offspring from poor development trajectories during the perinatal period. Archives of medical research, 47(1), 1-12.
4. Barker, D. J., et a., (1993). Fetal and infant origins of cardiovascular disease. Archives of Disease in Childhood, 68(6), 797.
5. Fichorova, R. N., et a., (2011). Maternal microbe-specific modulation of inflammatory response in extremely low gestational age newborns. MBio, 2(1), e00280-10.
6. Boney, C. M., et al., (2005). Metabolic syndrome in childhood: association with birth weight, maternal obesity and gestional diabetis mellitus. Pediatrics, 115(3), e290-e296.
7. Zambrano, E., et a., (2010). Dietary intervention prior to pregnancy reverses metabolic programming in male offspring of obese rats.

The Journal of physiology, 588(10), 1791-1799.
8. Desai, M., Crowther, N. J., Ozanne, S. E., Lucas, A., & Hales, C. N. (1995). Adult glucose and lipid metabolism may be programmed during fetal life.
9. Khan, I., et a., (2004). Predictive adaptive responses to maternal high-fat diet prevent endothelial dysfunction but not hypertension in adult rat offspring. Circulation, 110(9), 1097-1102.
10. Roseboom, T. J., (2001). The fetal origins hypothesis. Twin Res, 4(5), iii.
11. Samuelsson A. M., et a., (2008). Diet-induced obesity in female mice leads to offspring hyperphagia, adiposity, hypertension and insulin resistance; a novel murine model of developmental programming. Hypertension, 51(2), 383-392.
12. Rodriguez, J. S., et a., (2012). Maternal obesity in the rat programs male offspring exploratory, learning and motivation behavior: prevention by dietary intervention pre-gestation or in gestation. International Journal of Developmental Neuroscience, 30(2), 75-81.
13. Sanz, Y., et al., (2009). Gut microbiota and probiotics in modulation of epithelium and gut-associated lymphoid tissue function. International reviews of immunology, 28(6), 397-413.
14. Rautava, S., et al., (2012). Microbial contact during pregnancy, intestinal colonization and human disease. Nature Reviews Gastroenterology and Hepatology, 9(10), 565,576.
15. Vega, C. C., et al., (2015). Exercise in obese female rats has beneficial effects on maternal and male and female offspring metabolism. Int J Obes (Lond), 39(4), 712-719.
16. Rodriguez-Gonzalez, G. L., et al., (2015). Maternal obesity and overnutrition increase oxidative stress in male rat offsring reproductive system and decrease fertility. Int J Obes (Lond), 39(4), 549-556.
17. Rodriguez-Gonzelez, G. L., Castro-Rodriguez, D. C. & Zambrano, E. (2018). Pregnancy and Lactation: A Window of Opportunity to Improve Individual Health. 115-144.
18. Myhre, R. et al. (2010). Intake of probiotic food and risk of spontaneous preterm delivery. American Journal of Clinical Nutrition 93, 151-157.
19. Ilmonen J, Isolauri E, Poussa T, Laitinen K (2011) Impact of dietary counseling and probiotic intervention on maternal anthropometric measurements during and after pregnancy: a randomized placebo controlled trial. Clin Nutr 30:156-164.
20. Jafarnejad S, Saremi S, Jafarnejad F, Arab A (2016) Effects of a Multispecies Probiotic Mixture on Glycemic Control and Inflammatory Status in Women with Gestational Diabetes: A Randomized Controlled Clinical Trial. J Nutr Metab 2016:5190846.
21. Wickens K L, Barthow C A, Murphy R, Abels P R, Maude R M, Stone R et al (2017) Early pregnancy probiotic supplementation with Lactobacillus rhamnosus HN001 may reduce the prevalence of gestational diabetes mellitus: a randomised controlled trial. Br J Nutr 117:804-813.
22. Karamali M, Dadkhah F, Sadrkhanlou M, Jamilian M, Ahmadi S, Tajabadi-Ebrahimi M et al (2016) Effects of probiotic supplementation on glycaemic control and lipid profiles in gestational diabetes: A randomized, double-blind, placebo-controlled trial. Diabetes Metab 42:234-241.
23. Brantsaeter A L, Myhre R, Haugen M, Myking S, Sengpiel V, Magnus P et al (2011) Intake of probiotic food and risk of preeclampsia in primiparous women: the Norwegian Mother and Child Cohort Study. Am J Epidemiol 174:807-815.
24. Rautava S, Kainonen E, Salminen S, Isolauri E (2012) Maternal probiotic supplementation during pregnancy and breast-feeding reduces the risk of eczema in the infant. J Allergy Cin Immunol 130:1355-1360.
25. Luoto R, Kalliomaki M, Laitinen K, Isolauri E (2010) The impact of perinatal probiotic intervention on the development of overweight and obesity: follow-up study from birth to 10 years. Int J Obes 34:1531-1537.
26. Schabussova I, Hufnagl K, Tang M L, Hoflehner E, Wagner A, Loupal G et al (2012) Perinatal maternal administration of Lactobacillus paracasei NCC 2461 prevents allergic inflammation in a mouse model of birch pollen allergy. PloS one 7 (7): e40271.

### DETAILED DESCRIPTION OF FIGURES

FIG. 1. Fertility rate expressed as the number of pregnant/number of animals in the group. Where: C control diet (n=12/15); CP control diet+1×10¹⁰ CFU/mL L. mesenteroides SD23 (n=12/15); MO high fat diet (n=7/19); MOP high fat diet+1×10¹⁰ CFU/mL L. mesenteroides SD23 (n=18/25). “*” significant difference of the effect of the diet between C and MO, Different letters indicate the statistically significant differences between groups C and CP or MO and MOP, test chi square, p<0.05.

FIG. 2. A) Weight previous to pregnancy, arrow indicates days (90) where intervention with probiotic begins and B) maternal weight during pregnancy and lactation, where: (●)=C control diet (n=12); (∘)=CP control diet+1×10¹⁰ CFU/mL L. mesenteroides SD23 (n=12); (▾)=MO high fat diet (n=7); (A)=MOP high fat diet+1×10¹⁰ CFU/mL L. mesenteroides SD23 (n=15). “*” Statistically significant difference during pregnancy or lactation among C, CP vs MO, MOP. Media±EE; p<0.05. One way ANOVA, Tukey Test.

FIG. 3. Biochemical parameters of mother at the end of lactation: A) Glucose. B) Cholesterol. C) Triglycerides (TGS). Where: C control diet (n=12); CP control diet+1×10¹⁰ CFU/mL L. mesenteroides SD23 (n=12); MO high fat diet (n=7); MOP high fat diet+1×10¹⁰ CFU/mL L. mesenteroides SD23 (n=15). Different letters indicate statistically significant differences among the groups. Media±EE; p<0.05. One way ANOVA, Tukey test.

FIG. 4. Histology of small intestine of mothers at the end of lactation: villi length. Where: C control diet (n=8); CP control diet×1×10¹⁰ CFU/mL L. mesenteroides SD23 (n=8); MO high fat diet (n=7); MOP high fat diet+1×10¹⁰ CFU/mL L. mesenteroides SD23 (n=8). Different letters indicate statistically significant differences among the groups. Media±EE; p<0.05. One way ANOVA, Tukey test. Histological cuts H&E stained observed at 10× in small intestine villi.

FIG. 5. A) Weight of male offspring at 36 postnatal days; B) Total fat of male offspring at 36 postnatal days; C) Weight of female offspring at 36 postnatal days and D) Total fat of female offspring at 36 postnatal days. Where: C offspring of mothers with control diet; CP offspring of mothers intervened with control diet 1×10¹⁰ CFU/mL L. mesenteroides SD23; MO offspring of mothers with high fat diet; MOP offspring of mothers intervened with high fat diet+1×10¹⁰ CFU/mL L. mesenteroides SD23. C(n=8), CP(n=8), MO (n=7) and MOP(n=8) from different litter. Different letters indicate statistically significant differences among the groups. Media±EE; p<0.05. One way ANOVA, Tukey test.

FIG. 6. Biochemical parameters of offspring at 36 postnatal days; Male offspring: A) Glucose; B) Cholesterol; C) Triglycerides (TGS). Where: C offspring of mothers with control diet; CP offspring of mothers intervened with control diet+1×10¹⁰ CFU/mL L. mesenteroides SD23; MO offspring of mothers with high fat diet; MOP offspring of mothers intervened with high fat diet+1×10¹⁰ CFU/mL L. mesenteroides SD23. C(n=8), CP(n=8), MO(n=7) and MOP(n=8) from different litter. Different letters indicate statistically significant differences among the groups. Media±EE; p<0.05. One way ANOVA, Tukey test.

FIG. 7. A) Weight of male offspring at 110 postnatal days; B) Total fat of male offspring at 110 postnatal days; C) Adiposity index of male offspring at 110 postnatal days; D) Weight of female offspring at 110 postnatal days; E) Total fat of female offspring at 110 postnatal days and F) Adiposity index of female offspring at 110 postnatal days. Where: C offspring of mothers with control diet; CP offspring of mothers intervened with control diet+1×10¹⁰ CFU/mL L. mesenteroides SD23; MO offspring of mothers with high fat diet; MOP offspring of mothers intervened with high fat diet+1×10¹⁰ CFU/mL L. mesenteroides SD23. C(n=12), CP(n=11), MO(n=7) and MOP(n=15) from different litter. Different letters indicate statistically significant differences among the groups. Media±EE; p<0.05. One way ANOVA, Tukey test.

FIG. 8. Biochemical parameters of offspring at 110 postnatal days; Male offspring: A) Glucose; B) Cholesterol; C) Triglycerides (TGS); Female offspring: D) Glucose; E) Cholesterol and F) Triglycerides (TGS). Where: C offspring of mothers with control diet; CP offspring of mothers intervened with control diet+1×10¹⁰ CFU/mL L. mesenteroides SD23; MO offspring of mothers with high fat diet; MOP offspring of mothers intervened with high fat diet+1×10¹⁰ CFU/mL L. mesenteroides SD23. C(n=11), CP(n=10), MO(n=7) and MOP(n=9) from different litter. Different letters indicate statistically significant differences among the groups. Media±EE; p<0.05. One way ANOVA, Tukey test.

FIG. 9. Body composition of offspring at 350 postnatal days: Male offspring: A) Percentage of fat mass regarding body weight; B) Percentage of lean mass regarding body weight; C) Weight. Female offspring: D) Percentage of fat mass regarding body weight; E) Percentage of lean mass regarding body weight and F) Weight. Where: C offspring of mothers with control diet; CP offspring of mothers intervened with control diet+1×10¹⁰ CFU/mL L. mesenteroides SD23; MO offspring of mothers with high fat diet; MOP offspring of mothers intervened with high fat diet+1×10¹⁰ CFU/mL L. mesenteroides SD23. n=7 for all groups and they correspond to different litter. Different letters indicate statistically significant differences among the groups. Media±EE; p<0.05. One way ANOVA, Tukey test.

1.-4. (canceled)

5. A method of treating or preventing weight gain in offspring of mothers with high fat diet by administering an effective amount of a composition comprising Leuconostoc mesenteroides subsp. mesenteroides SD23 strain to an animal in need thereof.

6. The method according to claim 5, wherein the composition is a pharmaceutical composition.

7. The method according to claim 6, wherein the pharmaceutical composition is suitable to be administered in the form of a solid formulation, liquid formulation, or semi-solid or powder.

8. The method according to claim 6, wherein the pharmaceutical composition is suitable for parenteral, oral, colposcopy administration or rectal administration.

9. The method according to claim 5, wherein the composition is in the form of a food or beverage.

10. The method according to claim 5, wherein the animal is a human being.

11. A method of treating or preventing obesity in the offspring of mothers with high fat diet, by administering an effective amount of a composition comprising Leuconostoc mesenteroides subsp. mesenteroides SD23 strain to an animal in need thereof.

12. The method according to claim 11, wherein the composition is a pharmaceutical composition.

13. The method according to claim 12, wherein the pharmaceutical product is suitable to be administered in the form of a solid formulation, liquid formulation, or semi-solid or powder.

14. The method according to claim 12, wherein the pharmaceutical composition is suitable for parenteral, oral, colposcopy administration or rectal administration.

15. The composition according to claim 11, wherein the composition is in the form of a food or beverage.

16. The method according to claim 11, wherein to the animal is a human being.

17. A method of treating fertility problems in female rats fed with high fat diet, by administering an effective amount of a composition comprising Leuconostoc mesenteroides subsp. mesenteroides SD23 strain to a female rat in need thereof.

18. The method according to claim 17, wherein the composition is a pharmaceutical composition.

19. The method according to claim 18, wherein the pharmaceutical composition is in the form of a solid formulation, liquid formulation, or semi-solid or powder.

20. The method according to claim 18, wherein the pharmaceutical composition is suitable for parenteral, oral, colposcopy administration or rectal administration.

21. The method according to claim 17, wherein the composition is in the form of a food or beverage.

22. A method of treating high glucose, high cholesterol and high triglycerides concentrations in serum of female rats that consumed a high fat diet during the periods of gestation and lactation, by administering an effective amount of a composition comprising Leuconostoc mesenteroides subsp. mesenteroides SD23 strain to a female rat in need thereof.

23. The method according to claim 22, wherein the composition is a pharmaceutical composition.

24. The method according to claim 23, wherein the pharmaceutical composition is suitable to be administered in the form of a solid formulation, liquid formulation, or semi-solid or powder.

25. The method according to claim 23, wherein the pharmaceutical composition is suitable for parenteral, oral, colposcopy administration or rectal administration.

26. The method according to claim 22, wherein the composition is in the form of a food or beverage.
